(19) United States
(12) Patent Application Publication
(10) Pub. No.: US 2008/0147058 A1
(43) Pub. Date: Jun. 19, 2008

(54) Electrocautery system, provided with safe lighting during operational use

(76) Inventors: Robin S. Horrell, Billings, MT (US); David James Bibelhausen, Maineville, OH (US); Mickey M. Karram, Cincinatti, OH (US); John F. Love, Branchburg, NJ (US)

Correspondence Address: SYNNESTVEDT LECHNER & WOODBRIDGE LLP, P O BOX 592, 112 NASSAU STREET, PRINCETON, NJ, 08542-0592, US

(21) Appl. No.: 11/451,941

(22) Filed: Jun. 13, 2006

Related U.S. Application Data
(60) Provisional application No. 60/690,384, filed on Jun. 13, 2005.

(51) Int. Cl. A61B 18/12

(52) U.S. Cl. 606/37

(57) ABSTRACT

A cautery tool is provided with a compact, lightweight, user-directable lighting facility that can be powered by either a shared external power source from which a power flow is shared continuously with the cautery function, by a supplemental self-contained power cell that may be recharged by a portion of the external power for use independently of the cautery function, or by sharing of a self-contained power cell by the cautery and the lighting functions. Waste heat generated during exercise of the lighting function is continuously removed from a light-emitting element by electrical conductors also serving as heat transfer conduits.

### FIELD OF THE INVENTION

This invention relates to an electrocautery system that is provided with user-directed, lightweight, compact, optionally self-powered, safe lighting of an operational site at which an electrocautery operation is to be performed. More particularly, this invention relates to a self-contained, lightweight, compact, manually-handled electrocautery system provided with internally-cooled lighting directable by a user at an operational site, that is self-powered for both electrocautery and lighting needs.

### BACKGROUND OF THE RELATED ART

Safe, well-directed, adequate lighting of an operational site is essential during surgical procedures, both while a surgical tool is being applied to tissue and to enable the user to otherwise view and manipulate the tissue at and around the operational site. The carefully controlled application of a relatively high temperature to selected tissue, to effect an incision or local fusion cautery of the same, is the primary purpose of a cauterization procedure.

In a monopolar electrocautery system, this involves the application of a particularly shaped electrically conductive element at the end of a hand-piece, sometimes referred to as a “pencil”, to the tissue of a patient who is made a part of a shared electrical circuit. Alternatively, for incisions only, the hand-piece at a distal end may have an electrically heated thin wire stretched out tautly between two adjacent electrodes. In yet another alternative, in a bipolar system, tissue may be pressed between two cooperating electrodes movable relative to each other. In all of these alternatives, the user typically operates either a foot switch or a manual switch located on the pencil to cause a controlled flow of electrical current through the electrode(s). The electrode(s) will typically have a small thermal capacitance, and will therefore cool quite rapidly when the electrical current flow ceases.

The lighting portion of the system also heats up due to the release of waste heat while the user is cauterizing tissue and, often for relatively longer periods, while the user is manipulating tissue at and around the operational site. Local temperatures at or close to the light-generating element could consequently reach values high enough to cause damage to nearby tissue. Preferably, this heat must be removed on a more or less continual basis to limit the resulting temperature rise to eliminate the risk of inadvertent damage to adjacent tissue.

For prolonged surgical operations, particularly in confined regions, e.g., during gynecological or laryngeal surgery, it is very important for the user that both the physical size and the weight of the hand-held electrocautery element be limited to the extent possible. This means that there are serious physical limits on any means employed to effect heat transfer from the light-generating portion of the electrocautery system during its use, particularly when the light-generating element is mounted to the hand-held electrocautery element. Fans to cause forced convection of cooling airflow, or liquid cooling flows, therefore are generally impractical and intrusive when the user is operating within a confined body cavity of a patient.

Electrical power for cauterization and for lighting may be obtained from an electrical mains supply, via appropriately designed circuitry. Various circuits are commercially available, and electrical engineers of ordinary skill in the art may choose to design their own alternatives, to adapt any available electrical power supply to suit the needs of a chosen cautery element or lighting-element. Power for both needs simultaneously, or solely for lighting, may also be provided by self-contained power storage elements such as batteries, rechargeable cells or supercapacitors contained within or mounted to the body of the pencil.

The prior art provides numerous solutions, of varying effectiveness, both for the lighting needs and for cooling of the light-generating element. Examples of prior art relating to means for providing light during surgery include U.S. Pat. No. 2,029,487, of Kleine, titled “Illuminated Cautery Electrode” and U.S. Pat. No. 6,428,180, of Karram et al., titled “Surgical Illumination Device and Method of Use”. Examples of prior art relating to removal of heat from lighting elements include U.S. Pat. No. 6,709,128, of Gordon, titled “Curing System”, that employs a forced convection motor-driven fan; and U.S. Pat. No. 6,834,977, of Surhiro et al., titled “Light Emitting Device”, wherein conduction of heat along electrical conductors is suggested.

### SUMMARY OF THE INVENTION

It is a principal object of this invention to provide a lightweight, compact, cautery device in which a hand-held cauterization element is provided with a lighting element that can be readily employed by a user to clearly light an operation site within a confined region in a patient's body both during actual cauterization and while otherwise viewing and manipulating tissue.

Another object is to provide a surgeon with a lightweight, compact, self-powered, cautery device that is entirely self-contained and can provide clear lighting of an operation site within a confined region both during an actual procedure and while otherwise viewing and manipulating tissue at and about an operation site.

A related object is to provide, in an electrocautery system, a hand-held cauterization element that can operate on a single outside source of electrical mains power to operate both a cauterization electrode in a monopolar cauterization system and to provide the user safe and clear lighting of the operation site for prolonged periods both during actual cauterization procedures and otherwise.

An even further object of this invention is to provide, in a cautery system that utilizes laser light energy to effect incision and cauterization with a hand-held cauterization element, means for providing safe, clear lighting of an operational site within a confined region in a patient's body—both during an incision or cauterization procedure and also while the user is viewing, manipulating or otherwise operating on tissue.

These and other related objects of the invention are realized by providing a lightweight, compact, cautery device having a hand-held body, for selectively incising or cauterizing tissue at a lighted operational site that may lie within a confined region or cavity of a patient's body. The device comprises:
a first connection element for connecting to a first power source, to convey therefrom a first power flow for cauterization;
a second connection element for connecting to a second power source, to convey therefrom a second power flow for lighting;
a user-operable control circuit, connected to the first and second connection elements to enable a user to independently control said first and second power flows received therefrom;
a cautery element, mounted to a distal end of the body, connected by a first power conduit to receive the first controlled power flow from the control circuit and to generate heat as needed for said incision or cauterization;
a lighting-generating element, mounted to the body and connected by a second power conduit to receive the second controlled power flow from the control circuit independently of the first controlled power flow and to generate light directable by the user to the operational site; and
heat transfer means mounted to the body, in thermal communication with the lighting-generating element to receive and transfer away waste heat therefrom at a rate sufficient to limit the highest temperature of any tissue-contactable surface of the light-generating element to a predetermined safe value.

In another aspect of the invention, there is provided a method for enabling a user to safely direct clear lighting to an operational site from a hand-held cauterization tool both during an actual incision/cauterization procedure and while otherwise viewing, manipulating or operating within a confined region of a patient's body.

These and related objects are realized by providing a method for enabling safe, convenient, well-lit cautery, by application of a power-heated cautery blade mounted to a distal end of a hand-held body to tissue in a surgical operation, comprising the steps of:
providing a first flow of user-controlled power to heat the cautery blade;
providing an independent second flow of user-controlled power, via a power-conveying element connected to a lighting element mounted at the distal end of the body, to shine shadow-free light axially forward of the body at and about the tissue to be cauterized; and
transferring waste heat from the lighting element via the power-conveying element to a portion of the hand-held body serving as a heat sink, at a rate sufficient to ensure that the temperature of any tissue-contactable surface of the lighting element is always at a safe level.

### BRIEF DESCRIPTION OF THE DRAWINGS

FIG. 1A is a schematic view of a conventional monopolar cauterization arrangement, wherein controls for providing power to cauterize or make an incision are operated by a user's foot; and FIG. 1B is a schematic view of a similar arrangement wherein controls are operated by the user's fingers applied to buttons on the hand-held tool.

FIG. 2 is a schematic view of a conventional bipolar cauterization arrangement.

FIG. 3 is a perspective view of a hand-held surgical tool useful for cutting away tissue by application of heat thereto via a loop of heated wire extending distally forwardly of the tool.

FIGS. 4A and 4B are side elevation views of two versions of a hand-held cauterization tool according to this invention, wherein operating power is provided via an external cable.

FIGS. 5A-5D, respectively, are a top plan view, a side elevation view, a bottom plan view, and an end elevation view of a self-powered, hand-held, cauterization tool according to a preferred embodiment of this invention.

FIGS. 6A and 6B are two perspective views of the cauterization tool per FIGS. 5A-5B.

FIG. 7 is a circuit diagram suitable for a device that utilizes an external a.c. mains supply as its sole source of power both for monopolar cauterization/coagulation functions and for lighting of an operational site according to a preferred embodiment of this invention.

FIG. 8 is a circuit diagram suitable for a device according to this invention that utilizes an external a.c. mains supply as its sole source of power but also includes an auxilliary battery power source that can provide power for lighting independently of the power flow for the device's monopolar cauterization function.

FIG. 9 is a circuit diagram suitable for a device according to this invention that utilizes an external a.c. mains supply as its primary power source but has that supplemented by an auxiliary power d.c. flow that is separately used to power the lighting function independently of the cauterization function.

FIG. 10 is a basic schematic diagram showing the relationship of certain essential components of the lighting portion of this invention.

FIG. 11 is a circuit diagram of a known and suitable electrical circuit of a kind that enables realization of maximum battery life for a cauterization device according to a solely battery-powered embodiment.

FIG. 12 is a schematic pinout diagram for the circuit according to FIG. 11.

FIG. 13 is a block diagram clarifying details of the pinout portion of an integrated circuit according to FIG. 11.

FIG. 14 is a circuit diagram of a known and suitable circuit of a kind that enables realization of maximum brightness of LED light output from the lighting system according to this invention.

FIG. 15 is a circuit diagram of a known and suitable electrical circuit of a kind that enables optimum operation of a high power white light output LED for the lighting system according to this invention.

FIGS. 16A-16F are schematics of six optional types of wiring structures considered suitable for portions of the electrical circuits of the system according to this invention.

### DETAILED DESCRIPTION OF THE PREFERRED EMBODIMENTS

As best understood with reference to FIG. 1A, in a conventional arrangement for monopolar cauterization of a patient's tissue the patient “P” is placed on an electrically insulated surface 100 of an operating table 102. The patient is placed in contact with an electrically conductive contact pad 104 connected to a conductor 106, generally referred to as an electrical ground, which typically has a plug 108 at a distal end. The system has a control unit 110 that can be plugged into an electrical power source (not shown, but usually an a.c. mains socket) via plug 112 to receive an electric power flow, e.g., at 110V at 50 cps, via cable 114. Suitable circuitry of known kind (not shown), usually mostly accommodated in control unit 110, modifies the received current and voltage as appropriate. Control unit 110 has an output socket (not shown) into which a user can plug in a handpiece 116 via a cable 118 and plug 120. Handpiece 116 in such a system may have no additional controls, but at its forward end supports a small, deliberately shaped cautery blade 122.

The circuit inside control unit 110 is connected by a cable 124 to foot-operated switch unit 126 controlled by the application of foot pressure by the user. As illustrated in FIG. 1A, the switch unit 126 has two control pedals 128 and 130 that are disposed for easy access by the user standing close to the patient. Typically, with conventional systems of this kind, pedals 128 and 130 may be operated to deliver respective controlled power flows, at selected voltages and frequencies, to cautery blade 122 to effect corresponding cutting/incision or cautery/coagulation functions both of which require the local application of high temperature heat.

Cutting/incision typically requires a higher power flow, to generate a higher tissue-contact temperature than does cautery/coagulation for most tissues. The required heating of the monopolar cautery blade 122 to apply this heat is obtained by resistance heat released at the tissue contact point when blade 122 is contacted to the patient's tissue at a selected operational site to complete an electrical circuit through the patient's body. Cautery blade is typically shaped to effect cutting at an elongate edge that is not mechanically sharp like a knife but generates a relatively high current density by tissue contact at a small contact surface area. Heat-induced disintegration of the contacted tissue cells is probably a major factor that causes the cutting of tissue. The actual “cutting” mechanism no doubt also involves some local arcing between the blade and tissue because of the electrical potential differences—which would result in physical weakening and disintegration of tissue cell walls.

Cauterization of tissue usually requires a lower local temperature, obtained by reduced voltage and/or frequency of the current flow and by applying the heat and some mechanical pressure over a larger contact area. This is generally effected by applying the side of blade 122 to tissue to be fused by local heating at a temperature not sufficient to cause the level of cell disruption involved in cutting. Both cautery, e.g., of a severed artery, and coagulation of body fluids may be effected in this manner.

FIG. 1B shows an alternative arrangement in which foot switches 128 and 130 are replaced by hand-operated counterpart switches 178 and 180 on modified handpiece 166 equipped with cautery blade 122. Note that like parts are given the same identification numbers in both FIGS. 1A and 1B for ease of reference.

It is a combination of controlled power flows and user skill that provides the best results. Naturally, the user must be able to see the operational site clearly, i.e., clear, consistent and dependable lighting is very important. Logic dictates the lighting unit be optimally located on the handpiece, that the elements to provide it be small and light in weight, and that the light be bright and directable at the user's discretion both during an actual surgical procedure and otherwise while the user is viewing and/or manipulating tissue at and about the operational site. Since the power needs of such a lighting system generally are different from those of the blade 122, and yet another control is involved to operate it, the circuit becomes more complicated. Note that plug 170 is shown in FIG. 1B as having three pins, to indicate that if a lighting element is located on the handpiece it would require additional electric conduits to receive power from the control unit 110. One of the pins is to convey electric current sent on to the cautery blade 122, and the other two pins are for current to or from the two foot pedals 128 and 130 for cutting and coagulation. If a lighting element were receiving power from the wave generator then one or two additional pins likely would be needed for that current flow. Cable 118 would be correspondingly different, and then so would the related control circuit (not shown).

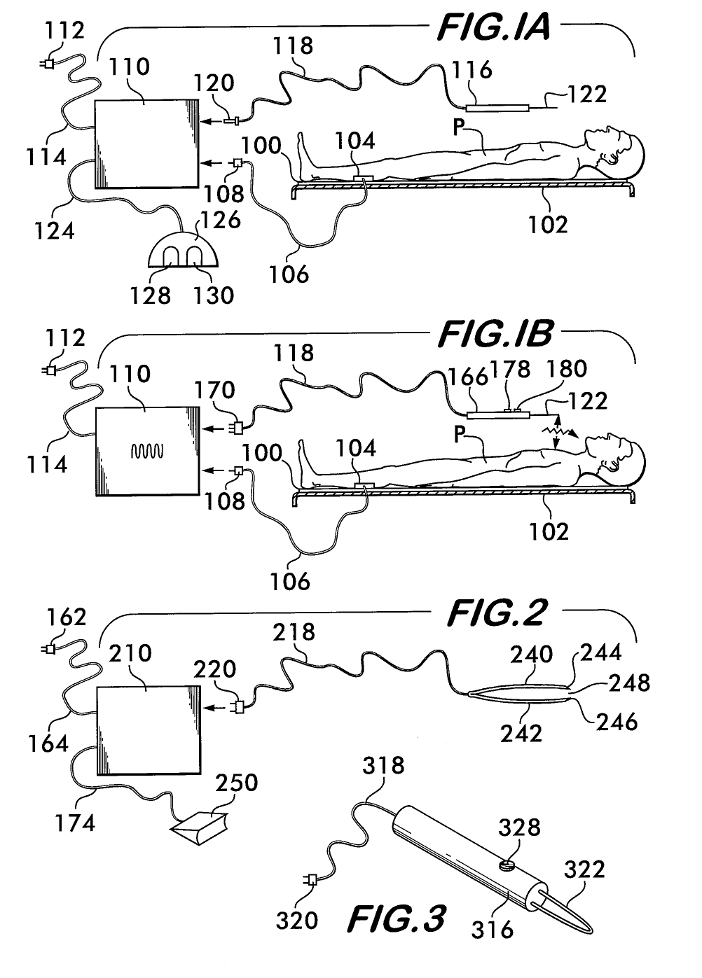

FIG. 2 presents a schematic view of a bipolar cautery system in which mains power, via control unit 210 is provided via plug 220 through cable 218 to contact terminals 240 and 242 that a user can move towards each other by hand to grasp tissue between their tips 244 and 246 in gap 248. Such contact pressure across the grasped tissue, with current flow obtained by the user actuating foot switch 250 would generate a controlled high temperature sufficient to cut through or to cauterize the tissue depending on the power employed. Obviously, the user's need for adequate lighting of the operational site remains the same.

FIG. 3 shows a third type of system, in which handpiece 316 receives electric power via cable 318 and plug 320 from an external power source to heat a distal loop 322 of a high resistance wire. Switch 328 allows the user to heat and apply the loop 322 to snag and then cut away tissue by burning through it locally. Such a device also could benefit from provision of adequate lighting.

Details of how the present invention addresses these needs follow below.

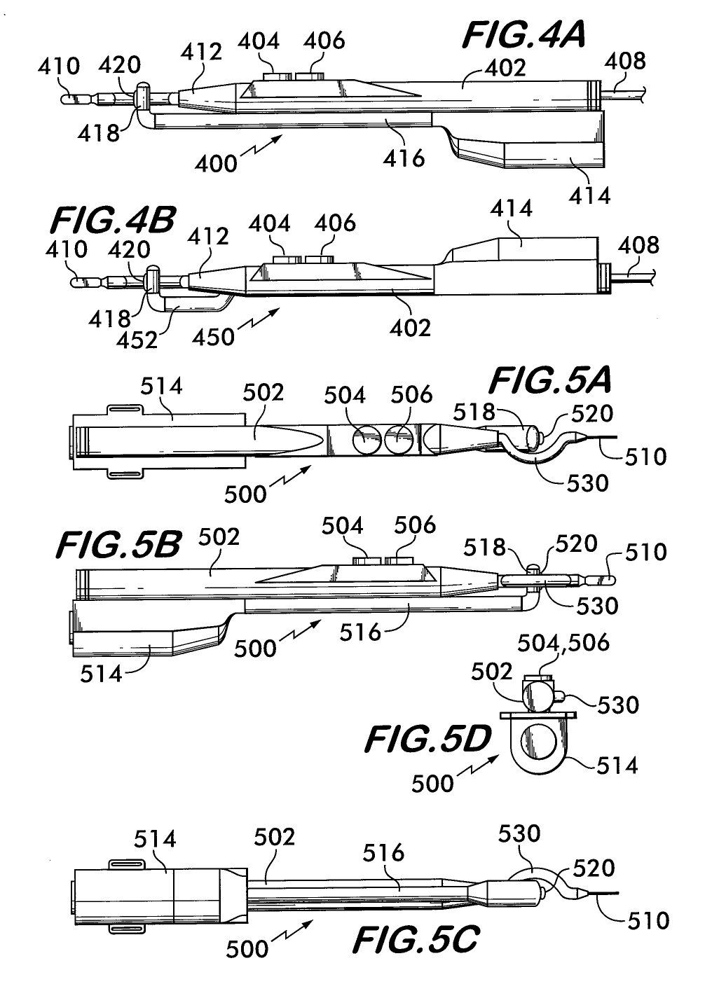

FIGS. 4A and 4B show side views of the first and second preferred embodiments, 400 and 450 respectively, of this invention. Both embodiments have an elongate hand-held body, equipped with two finger or thumb actuated switches 404 and 406, and are connectable to a power supply via a cable extending from the rear. Both embodiments also have an axially aligned cautery blade 410 extending forwardly from the front end, mounted to a base 412 which may be sealed to the body 402 by being snap-fitted or threaded thereto. Both embodiments further have a body-mounted power unit 414 adjacent the rear end to contain means to store power for powering a lighting portion of the handpiece.

First embodiment 400, per FIG. 4A, has an elongate lighting stalk 416 extending longitudinally along and outside the body 402 to support a light-emitting element 418, e.g., an LED, that has a lens 420 to project emitted light axially forward, along and past the cautery blade 410. The entire light-generating element, comprising power unit 414, light stalk 416, light-emitting element 418 and lens 420, in this embodiment could be detachably attached to body 402 in any of various known ways, e.g., by a snap-fitting, releasable adherent, Velcro™, or the like. Power unit 414 may contain a rechargeable cell (not shown) that can receive power from an external power supply (also not shown) via cable 408 to store up power to provide lighting when cauterization is not being effected, e.g., to allow the user to merely view and/or manipulate tissue. In practice, most users might use just the lighting feature in this manner for longer periods than they actually spend cauterizing or cutting. Alternatively, power unit 414 could contain a power cell (not shown) like a battery, a rechargeable cell or a supercapacitor and be independent of the external power supply. Power for cautery could be received via cable 408 and delivered via switch 404 or 406 (one being for the light and the other for cautery power flow). This would ensure that if the user actuates both switches 404 and 406 together, e.g., to have lighting while cauterizing, there would be no diminution or alteration of power delivered to the cautery blade 410 or to light-emitting element 420. These two power needs differ significantly, e.g., they require different voltages at different frequencies, hence selection of proper circuitry to control their delivery is very important.

The second embodiment, per FIG. 4B, differs from the first embodiment per FIG. 4B in at least one important respect—it does not have an elongate axially aligned light stalk. Instead, it has a shorter light stalk 452 extending at a small angle to the body close to the front end thereof. This results in a more compact handpiece that weighs less.

Note that stalks 416 or 452, power unit 414, light-emitting element 418, and even cable 408 can be made in known manner to be detachably fitted to the body and/or to each other as needed. This allows for the benefits of modularization, i.e., a manufacturer could produce such elements in various lengths, light-emitting capabilities, etc., and a user could easily fit together the assembly optimum for his or her intended use. Large surgical facilities might find this highly economical. It would also facilitate reuse of some or all or these components after cleaning, sterilization and reassembly—perhaps at reduced costs by workers abroad.

FIGS. 5A-5D are various views of a third embodiment in which the handpiece is entirely self-contained, it does not have or require a trailing cable to connect to an external power supply. The requisite power would in this case be stored in single-use or rechargeable power cells, batteries or supercapacitors located within power unit 514 that attaches to body 502 as previously discussed with respect to the first two embodiments. Many users might prefer the freedom resulting from elimination of the cable extending from the rear of the body, particularly for operations in highly confined regions. The two body-mounted switches 504 and 506 operate to individually control cautery and lighting power flows, the associated circuit being contained within lighting unit 514 and/or body 502.

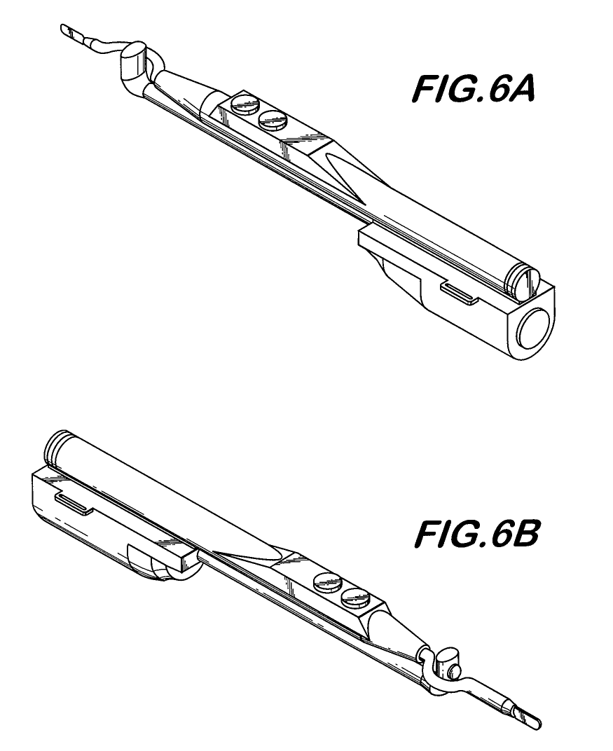

FIGS. 5A-5B clearly show a very important advantage of this invention over the prior art, particularly for monopolar devices. This is the manner in which the proximal portion 530 of the cautery blade element curves around and past the light-emitting element 518, so that light emitted forwardly from lens 520 projects axially along and past the distal portion of the blade element and cautery blade 510. This ensures that the tissue directly in front of the blade, and obviously tissue around and about it, will be lit without shadows. This benefit will be realized not only when blade 510 is applied, e.g., either to cut or to coagulate, but more often when the user wants to view tissue before or after an operation. The result will be improved precision and less stress for the user. FIGS. 6A and 6B make this feature very clear in perspective views. Note that his feature is present in both the cable-powered and the self-contained embodiments, e.g., those per FIGS. 4A and 4B.

The preferred light-emitting element is an LED that provides a white light, and has a forward bias voltage of 3.4V and requires a constant current of about 350 mA, i.e., a power requirement of about 1.2 W. While this is only a small fraction of the power required by the cautery blade (about two orders of magnitude larger) over a period of minutes it is possible for the light-emitting element to reach tissue-contactable surface temperatures high enough to cause serious tissue damage by inadvertent contact. There is also the danger that even the user might contact such a hot surface during the surgical operation and, despite wearing surgical gloves, might suffer pain and/or serious distraction. There is also concern about flammable items, e.g., small surgical drapes, alcohol soaked items and the like, becoming too hot unexpectedly, and about the presence of oxygen that is often required for patients and must be kept in the vicinity of the surgical zone. For all these reasons it is desirable to remove heat away from the light-emitting element as efficiently as possible. This must be done, to the extent possible, without adding to the size or weight of the handpiece and without increasing power requirements.

The most satisfactory solution to the above-referenced cooling problem is to utilize the already present elements wisely by making them do double duty whenever possible, e.g., make electrical conductors also serve as thermal conduits to transfer heat away from hot regions. This logic is applied beneficially in this invention as follows: the base that supports the light-emitting element (an LED, for example) has a certain mass and 6B make this feature very clear in perspective views. Note that his feature is present in both the cable-powered and the self-contained embodiments, e.g., those per FIGS. 4A and 4B.

The preferred light-emitting element is an LED that provides a white light, and has a forward bias voltage of 3.4V and requires a constant current of about 350 mA, i.e., a power requirement of about 1.2 W. While this is only a small fraction of the power required by the cautery blade (about two orders of magnitude larger) over a period of minutes it is possible for the light-emitting element to reach tissue-contactable surface temperatures high enough to cause serious tissue damage by inadvertent contact. There is also the danger that even the user might contact such a hot surface during the surgical operation and, despite wearing surgical gloves, might suffer pain and/or serious distraction. There is also concern about flammable items, e.g., small surgical drapes, alcohol soaked items and the like, becoming too hot unexpectedly, and about the presence of oxygen that is often required for patients and must be kept in the vicinity of the surgical zone. For all these reasons it is desirable to remove heat away from the light-emitting element as efficiently as possible. This must be done, to the extent possible, without adding to the size or weight of the handpiece and without increasing power requirements.

The most satisfactory solution to the above-referenced cooling problem is to utilize the already present elements wisely by making them do double duty whenever possible, e.g., make electrical conductors also serve as thermal conduits to transfer heat away from hot regions. This logic is applied beneficially in this invention as follows: the base that supports the light-emitting element (an LED, for example) has a certain mass that can serve as a thermal mass, heat capacitance or heat sink, i.e., it will absorb some of the heat from the LED to cool it temporarily. It will be especially effective in this if it can shed some of the collected heat, e.g., by conducting it to electrical conductors touching it. This is to some extent inherent in the structure, but designing the base and the conductors with this in mind, and selecting materials that are particularly good thermal and electrical conductors significantly enhances this benefit. The electrical conductor so employed to do double duty will also serve as a second sink for limited periods—clearly beneficial to the cooling effort. Even further, the far end of electrical conductor so employed will be able to transfer some of the conducted heat to the power unit, e.g., to the mass of the power cell if there is one. Eventually, the user's own hand will absorb some of the transferred heat away from the handpiece. Accordingly, the conductors contacting the light-emitting element base, and the base itself, are preferably contain at least one of aluminum, copper, gold, brass, beryllium-copper alloy, platinum and titanium. These materials are considered good electrical and thermal conductors, and there may be others that would qualify equally well.

Note that a number of cautery systems utilize laser light energy to effect cutting by direct application of laser light to the tissue to cause intense local heating thereof. A variation of this is to absorb the laser light internally at the surface in a thin external coating on the cautery blade to heat the coated surface and apply it for cautery or coagulation. Even such systems can be improved for use in confined regions by supplementing them with the cooled lighting system taught herein. The electrical conductors that convey power, e.g., from a self-contained power source such as a single-use or rechargeable cell in or on the handpiece, can also be adapted to help in removing heat from the light-emitting element as taught herein.

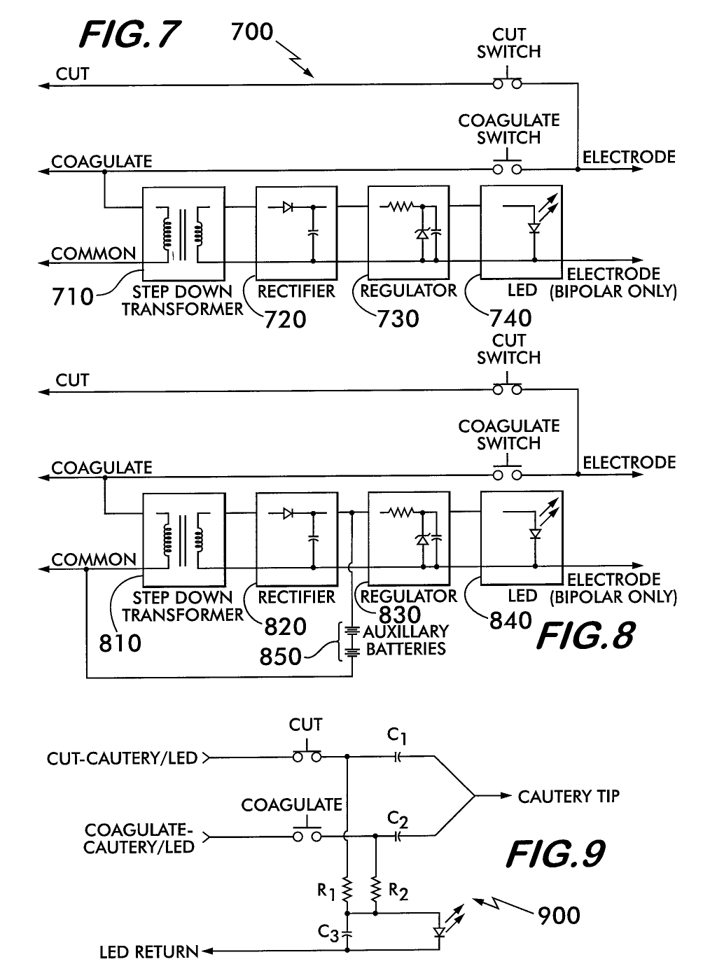

FIG. 7 is a circuit diagram of a circuit 700 considered particularly suitable for inclusion in the control circuit of an embodiment that receives electric power from an a.c. mains supply, uses most of it for the cautery function and a relatively small amount for the lighting function. It is initially necessary to modify the voltage and the frequency of the received power flow to suit the cautery function needs. This is done in what is commonly called a wave generator. Typical commercially available systems rate at about 160 W for coagulation and about 290 W for cutting purposes. It is proposed to “scavenge” some of this modified power output and further modify it for the lighting function. Physically, this requires that in a monopolar cautery system an electrical return path be provided for the current flow back from the LED. Circuit 700, per FIG. 7, is intended to do this.

The nominal voltage of the signals delivered from the electrocautery power supply to the electrodes is greater than that required or directly usable for LED power. A step down transformer 710 is therefore used to modify the voltage. The scavenged signal from the transformer 710 is then rectified and filtered in rectifier 720, and the rectified output is converted to the appropriate lower voltage in regulator 730 and then provided to LED 740. As will be understood by persons of ordinary skill in the electrical arts, this will allow the user to operate both the cautery and the lighting elements via the switches on the handpiece. The former could be a two-position type that would allow selection of the correct power for cutting or coagulating as needed.

It may be highly useful to provide a supplementary power source in the handpiece, e.g., a battery, to power just the lighting function for lighting when the power otherwise scavenged from the cautery power flow is not available. Circuit 800, per FIG. 8, is intended to do this. It differs from the circuit per FIG. 7 in that it includes auxiliary batteries. These may be small in size and weight, being needed only for limited duty, i.e., to run the LED for a short time.

Yet another option is to add to the cautery signal a supplemental power flow and then strip it away for use in the lighting function. Such a supplemental power flow could be in the nature of a direct current addition to the primary alternating current flow. This will require the addition of two more conductors to provide the necessary electrical pathways in the circuit. Circuit 900, per FIG. 9, is intended to do this. There are various ways to separate the two signals, a.c. and d.c., that are transmitted on the same conductor. One preferred method is to use a simple passive electronic lowpass filter to remove the a.c. component from the d.c. component; and use a capacitor in series with the cautery signal to block the d.c. component. Other obvious variations will no doubt occur to those skilled in the relevant art, and all such are intended to be comprehended within the scope of this disclosure.

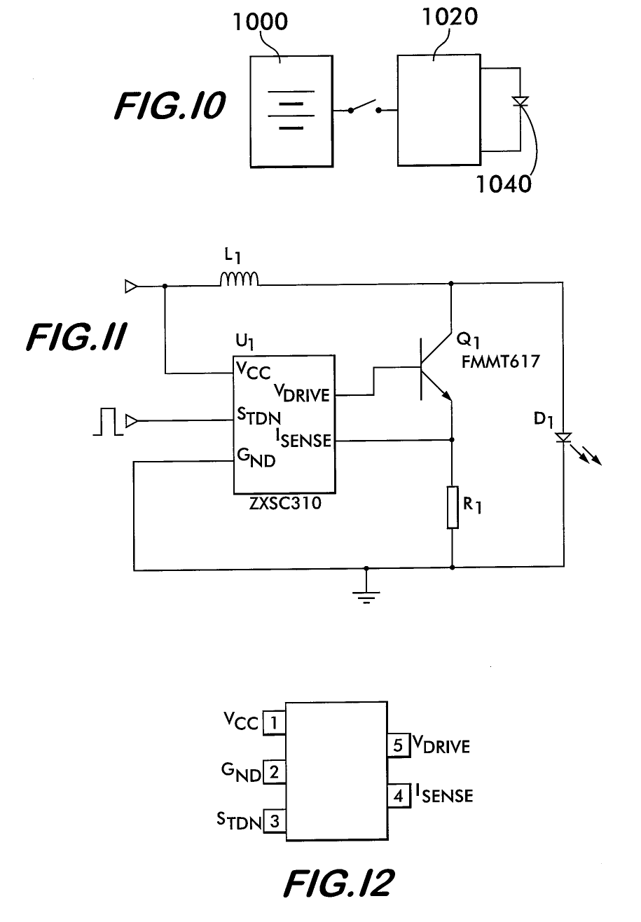

FIG. 10 is a basic schematic diagram that identifies key portions of the electrical circuit of the lighting portion. These are: the power source portion 1000, the power conversion portion 1020 and the light-producing portion 1040, respectively. The power source portion 1000, especially in an embodiment with a self-contained handpiece, will contain at least one power cell. This may be a single-use or rechargeable battery selected for its ability to deliver at a reasonably steady voltage for consistent lighting. Provision of the correct voltage to the LED is accomplished by the conversion portion 1020 which converts the energy flow from the power source portion to a constant pulsed current or a constant direct current at a voltage required to forward bias the semiconductor junction in the LED. A preferred power source is a single lithium battery cell with a nominal 3.2V rating, especially for a cautery system with a totally sealed handpiece. It is also suitable for modular systems because upon exhaustion only the power source portion needs to be replaced. Another option is to use a rechargeable cell like those used in computers, cell phones, DVD players, etc. These have a long life but eventually have to be replaced. Despite their higher initial costs, economies of scale may make them preferable for large users like hospitals or emergency centers.

The power conversion portion 1020 performs a voltage boost function, necessary because most suitable white light LEDs have a forward bias voltage higher than the 3.2V nominal voltage of a lithium cell. It must provide a constant voltage to ensure steady, clear and consistent lighting regardless of any decline in the output voltage from the power cell(s) as the energy stored therein is depleted to exhaustion. The power conversion portion 1020 preferably is based on a commercially available device marketed as a ZETEX ZXSC310. This is an integrated circuit which, when combined with a high performance external transistor, enables the production of a high efficiency boost converter for LED driving operations from a battery cell power source. Details of the ZETEX device, and certain variations thereof, may be found in ZETEX Semiconductors Bulletin, Issues 2 and 3, for March 2004. Some of the exemplary circuits are identified as “Prior Art” in FIGS. 11-15 hereof and are briefly described below.

FIG. 11 is a ZETEX circuit designed for maximum battery life in use. The LED in such an application is provided with a pulsed current.

FIG. 12 is an enlarged view of the pinout element identified as “U1” in FIG. 11, and explains the part of the circuit that engages with a single power cell.

FIG. 13 is a block diagram of the controller integrated circuit (IC) which in combination with a high performance external transistor drives the LED.

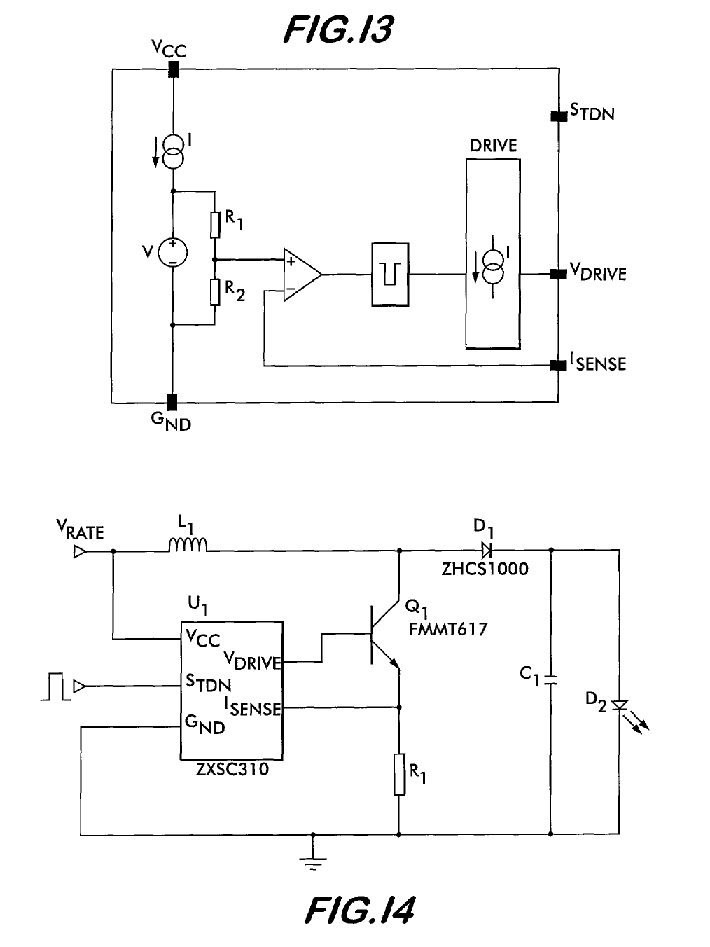

FIG. 14 is a modified circuit that provides a maximum brightness solution by rectifying and buffering the DC-AC output made available to drive the LED.

FIG. 15 shows the ZETEX ZXSC310 as configured to drive a 1 W LED that has a 180 CD light output from a forward current of 350 mA, and the power source comprises two cells.

As noted earlier, employment of electrical conductors and heat sink masses constitutes efficient use of the mass and volume of the lighting system itself to ensure against unacceptably high temperature damage to inadvertently contacted tissues. Referring to FIG. 10, it should be understood the circuitry can be made very compact and rugged and that it may be located almost anywhere in the cautery system, e.g., within the power source (wave generator) 1000. It could similarly be located within the power conversion portion 1020 that might nbe placed within the handpiece, or even along any of the power cables.

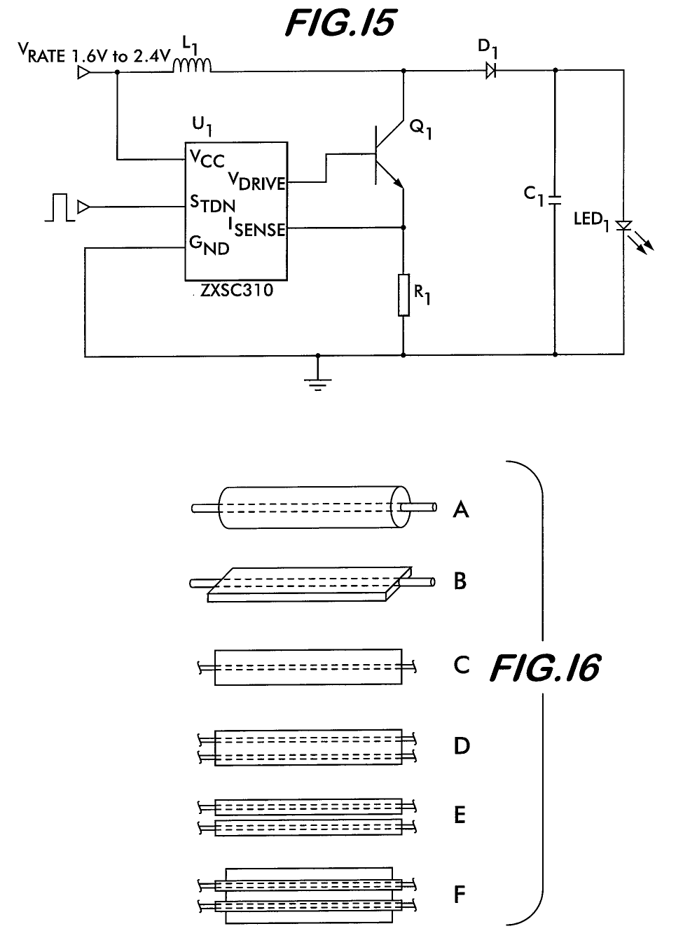

Various structural options are available in selecting the electrical conductors, some of which are indicated in FIGS. 16A-16F. Per FIG. 16A, for example, a single insulated wire is disposed within a malleable conductor serving as an enveloping sheath—particularly suitable for embodiments having a selectively deformable longitudinal stalk supporting the light-emitting element. A user can manipulate such a malleable stalk to direct light to suit particular needs.

Other similar flexible and malleable choices include:

per FIGS. 16B and 16C, an insulated electrical wire attached to a flat insulated conductor that will serve as the principal heat conduit;

per FIG. 16D, two insulated wire conductors attached closely to a thermally conductive element that will provide the principal thermal path for heat removal;

per FIG. 16E, two physically separate insulated conductors that may be twisted about the longitudinal stalk by a user to modify the lighting delivery to suit personal preferences; and

per FIG. 16F, two parallel insulated wires attached to an adherent tape that can be used to dispose them along the stalk by a user as needed.

Persons of ordinary skill in the relevant arts will no doubt consider and employ other obvious variations of the structures disclosed and suggested herein. All such modifications and variations are intended to be comprehended within this invention which is limited only by the appended claims.

1. A lightweight, compact, cautery device having a hand-held body, for selectively incising or cauterizing tissue at a lighted operational site, comprising:
a first connection element for connecting to a first power source, to convey therefrom a first power flow for cauterization;
a second connection element for connecting to a second power source, to convey therefrom a second power flow for lighting;
a user-operable control circuit, connected to the first and second connection elements to enable a user to independently control said first and second power flows received therefrom;
a cautery element, mounted to a distal end of the body, connected by a first power conduit to receive the first controlled power flow from the control circuit and to generate heat as needed for said incision or cauterization;
a lighting-generating element, mounted to the body and connected by a second power conduit to receive the second controlled power flow from the control circuit independently of the first controlled power flow and to generate light directable by the user to the operational site; and
heat transfer means mounted to the body, in thermal communication with the lighting-generating element to receive and transfer away waste heat therefrom at a rate sufficient to limit the highest temperature of any tissue-contactable surface of the light-generating element to a predetermined safe value.

2. The device according to claim 1, wherein:
the first power source comprises a mains power supply; and
the second power source comprises the same mains power supply.

3. The device according to claim 2, further comprising:
a rechargeable power storage unit, mounted to the body, that stores power received from said mains power supply and provides said second power flow from the stored power independently of the first power flow.

4. The device according to claim 2, wherein:
the circuit comprises a foot-operated switch to control the first power flow and a hand-operated switch mounted to the body to control the second power flow.

5. The device according to claim 2, wherein:
the circuit comprises first and second hand-operated switches, both mounted to the body, to enable a user to independently control the first and second power flows by hand.

6. The device according to claim 1, wherein:
a self-contained common power source, mounted to the body, comprises both the first and second power sources.

7. The device according to claim 6, wherein:
the common power source comprises a power storage unit selected from a group consisting of a single use power cell, a rechargeable cell and a supercapacitor.

8. The device according to claim 1, wherein:
the cautery element comprises a cautery blade extending axially forward from the distal end of the body.

9. The device according to claim 8, wherein:
the cautery element comprises a bypass portion intermediate a base and the cautery blade, the bypass portion being oriented to bypass the light-generating element so that the cautery blade is disposed coaxially with the body and forwardly of the light-generating element.

10. The device according to claim 8, wherein:
the light-generating element emits light forwardly of the distal end of the body, and the cautery element is disposed relative to the light-generating element so as to enable the emitted light to illuminate the cautery blade free of shadows.

11. The device according to claim 1, wherein:
the light-generating element comprises at least one light emitting diode (LED).

12. The device according to claim 1, wherein:
the heat transfer means comprises a base portion that supports the light-generating element and serves as a first heat sink for temporarily storing the waste heat.

13. The device according to claim 11, wherein:
the heat transfer means comprises a portion of the second power conduit that also functions as a heat conduit to enable transfer of waste heat from the first heat sink to the second power source that also functions as a second heat sink.

14. The device according to claim 1, wherein:
the first power source provides alternating electrical current at mains voltage and frequency; and
the control circuit comprises a first portion that modifies the mains voltage and frequency to respective values appropriate for the cautery element.

15. The device according to claim 13, wherein:
the second power source provides alternating electrical current at mains voltage and frequency; and
the control circuit comprises a second portion that modifies the mains voltage and frequency to respective values appropriate for the light-generating element.

16. The device according to claim 1, wherein:
the first power source comprises a source of laser light energy, and the first power flow is a flow of the laser light energy.

17. The device according to claim 13, further comprising:
a rechargeable power unit, mounted to the body;
wherein
the second power source provides electrical current at mains voltage and frequency, and
the control circuit comprises a charging portion that utilizes the electrical current from the second power source to charge the rechargeable power unit and to provide an output therefrom as appropriate for the light-generating element.

18. The device according to claim 6, wherein:
the heat transfer means comprises a base portion that supports the light-generating element and serves as a first heat sink for temporarily storing the waste heat; and
the heat transfer means further comprises a portion of the second power conduit that also functions as a heat conduit to enable transfer of waste heat from the first heat sink to the second power source that also functions as a second heat sink.

19. The device according to claim 6, wherein:
the control circuit comprises first and second portions that modify a current and a voltage provided by the common power source to respective values appropriate for the cautery element and the light-generating element.

20. The device according to claim 12, wherein:
the first and second heat sinks each comprise a material selected from a group of materials consisting of aluminum, copper, gold, brass, beryllium-copper alloy, platinum and titanium.

21. A method of providing safe, convenient, well-lit cautery, by application of a power-heated cautery blade mounted to a distal end of a hand-held body to tissue in a surgical operation, comprising the steps of:
providing a first flow of user-controlled power to heat the cautery blade;
providing an independent second flow of user-controlled power, via a power-conveying element connected to a lighting element mounted at the distal end of the body, to shine shadow-free light axially forward of the body at and about the tissue to be cauterized; and
transferring waste heat from the lighting element via the power-conveying element to a portion of the hand-held body serving as a heat sink, at a rate sufficient to ensure that the temperature of any tissue-contactable surface of the lighting element is always at a safe level.
